(19) United States
(12) Patent Application Publication
(10) Pub. No.: US 2015/0376135 A1
(43) Pub. Date: Dec. 31, 2015

(54) PROCESS FOR THE PREPARATION OF BIS-DIHALOALKYL PYRAZOLES

(71) Applicant: SYNGENTA PARTICIPATIONS AG, Basel (CH)

(72) Inventors: Anup JAWALEKAR, Goa (IN); Helmars SMITS, Stein (CH); Laura QUARANTA, Stein (CH); Sitaram PAL, Goa (IN); David Anthony JACKSON, Muenchwilen (CH)

(73) Assignee: Syngenta Participations AG, Basel (CH)

(21) Appl. No.: 14/763,921

(22) PCT Filed: Feb. 7, 2014
(86) PCT No.: PCT/EP2014/052455

§ 371 Date: Jul. 28, 2015

(30) Foreign Application Priority Data
Feb. 15, 2013 (IN) 442/DEL/2013

(51) Int. Cl. C07D 231/12; C07C 49/16; C07C 45/63

(52) U.S. Cl. CPC C07D 231/12; C07C 45/63; C07C 49/16

(57) ABSTRACT

The present invention relates to a process for the preparation of bisdihaloalkyl pyrazoles of formula (V) starting from diketones and acylhalides reacted with a Lewis acid, and a subsequent reaction with a substituted hydrazine.

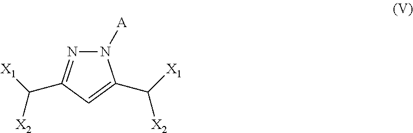

The present invention relates to a process for the preparation of bis-dihaloalkyl pyrazoles starting from diketones and acyl halides reacted with a Lewis acid, and a subsequent reaction with a substituted hydrazine. The present invention also relates to novel bis-dihaloalkylpyrazoles useful for the synthesis of fungicides as described in WO2013/000941.

The present invention relates to a process comprising at least the following steps:
reacting a compound of formula (I), with a compound of formula (II), in the presence of a Lewis acid to obtain a compound of formula (III):

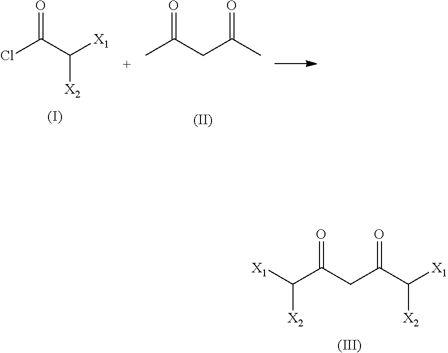

wherein X₁ and X₂ each independently are halogen, then
reacting the compound of formula (III) with a compound of formula (IV) to obtain a compound of formula (V):

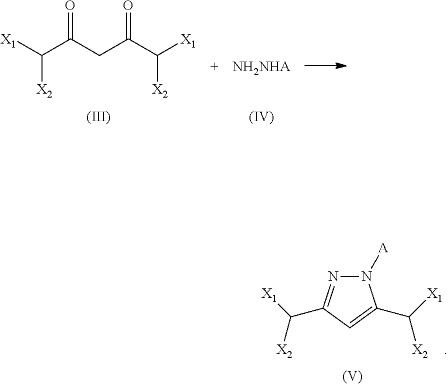

wherein,
A is hydrogen, C₁-C₆alkyl, C₁-C₆haloalkyl, C₁-C₆alcoxycarbonylC₁-C₆alkyl, hydroxycarbonylC₁-C₆alkyl, C₁-C₆alkenyl, C₁-C₆alkynyl, —C(═O)R^{A}, C₁-C₆alkylaryl, and
R^{A} is hydrogen, C₁-C₆alkyl, C₁-C₆alcoxy, C₁-C₆haloalkyl, NH₂, C₁-C₆alkylamino, C₁-C₆dialkylamino or one of the following groups:

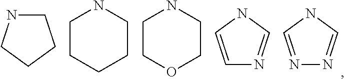

and X₁ and X₂ each independently are halogen.

Where substituents are indicated as being optionally substituted, this means that they may or may not carry one or more identical or different substituents, e.g. one to five substituents, e.g. one to three substituents. Normally not more than three such optional substituents are present at the same time. Where a group is indicated as being substituted, e.g. alkyl, unless stated otherwise this includes those groups that are part of other groups, e.g. the alkyl in alkylthio.

The term “halogen” refers to fluorine, chlorine, bromine or iodine, preferably fluorine, chlorine or bromine.

Alkyl substituents may be straight-chained or branched. Alkyl on its own or as part of another substituent is, depending upon the number of carbon atoms mentioned, for example, methyl, ethyl, n-propyl, n-butyl, n-pentyl, n-hexyl and the isomers thereof, for example, iso-propyl, iso-butyl, sec-butyl, tert-butyl, iso-amyl or pivaloyl.

Alkenyl substituents can be in the form of straight or branched chains, and the alkenyl moieties, where appropriate, can be of either the (E)- or (Z)-configuration. Examples are vinyl and allyl. The alkenyl groups are preferably C₂-C₆, more preferably C₂-C₄ and most preferably C₂-C₃ alkenyl groups.

Alkynyl substituents can be in the form of straight or branched chains. Examples are ethynyl and propargyl. The alkynyl groups are preferably C₂-C₆, more preferably C₂-C₄ and most preferably C₂-C₃ alkynyl groups.

Haloalkyl groups may contain one or more identical or different halogen atoms and, for example, may stand for CH₂Cl, CHCl₂, CCl₃, CH₂F, CHF₂, CF₃, CF₃CH₂, CH₃CF₂, CF₃CF₂ or CCl₃CCl₂.

Haloalkenyl groups are alkenyl groups, respectively, which are substituted with one or more of the same or different halogen atoms and are, for example, 2,2-difluorovinyl or 1,2-dichloro-2-fluoro-vinyl.

Haloalkynyl groups are alkynyl groups, respectively, which are substituted with one or more of the same or different halogen atoms and are, for example, 1-chloro-prop-2-ynyl.

Alkoxy means a radical —OR, where R is alkyl, e.g. as defined above. Alkoxy groups include, but are not limited to, methoxy, ethoxy, 1-methylethoxy, propoxy, butoxy, 1-methylpropoxy and 2-methylpropoxy.

Cyano means a CN group.

Amino means an NH₂ group.

Hydroxyl or hydroxy stands for a —OH group.

Aryl means a ring system which may be mono-, bi- or tricyclic. Examples of such rings include phenyl, naphthalenyl, anthracenyl, indenyl or phenanthrenyl. A preferred aryl group is phenyl.

Heteroaryl stands for aromatic ring systems comprising mono-, bi- or tricyclic systems wherein at least one oxygen, nitrogen or sulfur atom is present as a ring member. Monocyclic and bicyclic aromatic ring systems are preferred, monocyclic ring systems are more preferred. For example, monocyclic heteroaryl may be a 5- to 7-membered aromatic ring containing one to three heteroatoms selected from oxygen, nitrogen and sulfur, more preferably selected from nitrogen and sulfur. Bicyclic heteroaryl may be a 9- to 11-membered bicyclic ring containing one to five heteroatoms, preferably one to three heteroatoms, selected from oxygen, nitrogen and sulfur. Examples are furyl, thienyl, pyrrolyl, imidazolyl, pyrazolyl, thiazolyl, isothiazolyl, oxazolyl, isoxazolyl, oxadiazolyl, thiadiazolyl, triazolyl, tetrazolyl, pyridyl, pyridazinyl, pyrimidinyl, pyrazinyl, triazinyl, tetrazinyl, indolyl, benzothiophenyl, benzofuranyl, benzimidazolyl, indazolyl, benzotriazolyl, benzothiazolyl, benzoxazolyl, imiazothiazoyl, quinolinyl, quinoxalinyl, isoquinolinyl, phthalazinyl, quinoxalinyl, quinazolinyl, cinnolinyl and naphthyridinyl, preferably pyridyl, pyrazinyl, pyridazinyl, pyrimidinyl, pyrrolyl, pyrazolyl, imidazolyl, triazolyl, furanyl, thienyl thiazolyl or thiadiazolyl. Heteroaryl rings do not contain adjacent oxygen ring atoms, adjacent sulfur ring atoms or adjacent oxygen and sulfur ring atoms. A link to a heteroaryl group can be via a carbon atom or via a nitrogen atom.

Carbocyclic ring system includes aryl and in addition their saturated or partially unsaturated analogues.

Heterocyclyl and heterocyclic ring system are used interchangeably and unless otherwise stated refer to include heteroaryl and in addition their saturated or partially unsaturated analogues. The different rings of bi- or tricyclic heterocyclic ring systems may be linked via one atom belonging to two different rings (spiro), via two adjacent ring atoms belonging to two different rings (annelated) or via two different, not adjacent ring atoms belonging to two different rings (bridged).

The presence of one or more possible asymmetric carbon atoms in compounds of formula III and V means that the compounds may occur in optically isomeric forms, i.e. enantiomeric or diastereomeric forms. Also atropisomers may occur as a result of restricted rotation about a single bond. Formulas (III), (V), (Va), (Vb) and (Vc) are intended to include all those possible isomeric forms and mixtures thereof. The present invention includes all those possible isomeric forms and mixtures thereof for compounds of formulas (III), (V), (Va), (Vb) and (Vc). Likewise, formulas (III), (V), (Va), (Vb) and (Vc) are intended to include all possible tautomers. The present invention includes all possible tautomeric forms for compounds of Formulas (III), (V), (Va), (Vb) and (Vc).

In each case, the compounds disclosed in the process according to the invention are in free form, in oxidized form as a N-oxide or in salt form, e.g. an agronomically usable salt form.

N-oxides are oxidized forms of tertiary amines or oxidized forms of nitrogen containing heteroaromatic compounds. They are described for instance in the book “Heterocyclic N-oxides” by A. Albini and S. Pietra, CRC Press, Boca Raton 1991.

The following list provides definitions, including preferred definitions, for substituents X₁, X₂, X₃, X₄, X₅, X₆, X₇, A, R^{A}, B, and R^{B} with reference to compounds (I), (II), (III), (IV), (V), (Va), (Vb), (Vc), (VI), and other compounds of the invention carrying the same substituents. For any one of these substituents, any of the definitions given below may be combined with any definition of any other substituent given below or elsewhere in this document.

X₁ and X₂ each independently are halogen.

A is hydrogen, C₁-C₆alkyl, C₁-C₆haloalkyl, C₁-C₆alcoxycarbonyl C₁-C₆alkyl, hydroxycarbonyl C₁-C₆alkyl, C₁-C₆alkenyl, C₁-C₆alkynyl, —C(═O)R^{A}, C₁-C₆alkylaryl. R^{A} is hydrogen, C₁-C₆alkyl, C₁-C₆alcoxy, C₁-C₆haloalkyl, NH₂, C₁-C₆alkylamino, C₁-C₆dialkylamino or one of the following groups:

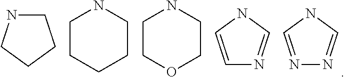

As mentioned above X₁ and X₂ each independently are halogen.

Preferably, X₁ and X₂ each independently are bromo, chloro or fluoro, and more preferably chloro or fluoro.

Preferably, A is C₁-C₆alkyl, C₁-C₆haloalkyl, C₁-C₆alcoxycarbonyl C₁-C₆alkyl, hydroxycarbonyl C₁-C₆alkyl, C₁-C₆alkenyl, C₁-C₆alkynyl, —C(═O)R^{A}, C₁-C₆alkylaryl.

In one embodiment A is C₁-C₆alkylaryl, with aryl being phenyl optionally substituted with halo, C₁-C₆alcoxy, NO₂, cyano, or C₁-C₆alkyl.

In an embodiment A is hydrogen.

In another embodiment A is —CH₂CO₂R, with R being hydrogen or C₁-C₄ alkyl, preferably methyl or ethyl.

B is C₁-C₆alkyl, C₁-C₆haloalkyl, C₁-C₆alcoxycarbonyl C₁-C₆alkyl, hydroxycarbonyl C₁-C₆alkyl, C₁-C₆alkenyl, C₁-C₆alkynyl, —C(═O)R^{B}, C₁-C₆alkylaryl, and R^{B} is hydrogen, C₁-C₆alkyl, C₁-C₆alcoxy, C₁-C₆haloalkyl, NH₂, C₁-C₆alkylamino, C₁-C₆dialkylamino or one of the following groups:

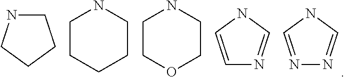

In one embodiment B is C₁-C₆alkylaryl, with aryl being phenyl optionally substituted with halo, C₁-C₆alcoxy, NO₂, cyano, or C₁-C₆alkyl.

In one embodiment B is H

In another embodiment B is —CH₂CO₂R, with R being hydrogen or C₁-C₄ alkyl, preferably methyl or ethyl

In a preferred embodiment, the process according to the invention comprises at least the following additional step:
reacting a compound of formula (V) with a fluoride anion source, to obtain a compound of formula (Va):

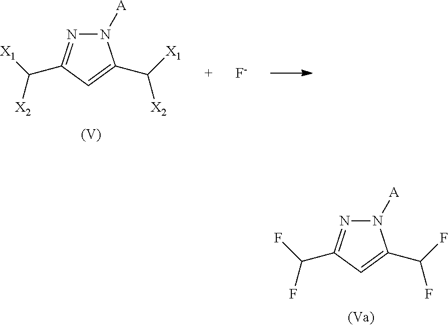

wherein A is as defined above,
X₁ and X₂, each independently are halogen, provided that at least one of X₁ and X₂ is not fluoro, and preferably X₁ and X₂, each independently are bromo or chloro.

In one embodiment of the invention, the process according to the invention comprises at least the following additional step:
reacting a compound of formula (V) with a fluoride anion source, to obtain one or several compounds of formula (Vb):

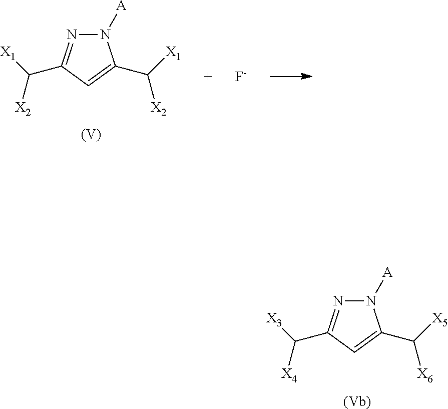

wherein A is as defined above,
X₁ and X₂, each independently are halogen, provided that at least one of X₁ and X₂ is not fluoro, and preferably X₁ and X₂, each independently are bromo or chloro,
X₃, X₄, X₅ and X₆ each independently are halogen, provided that at least one of X₃, X₄, X₅ and X₆ is fluoro,
optionally reacting compounds of formula (Vb) one or several times, and preferably 1, 2, 3 or 4 times with a fluoride anion source to obtain a compound of formula (Va)

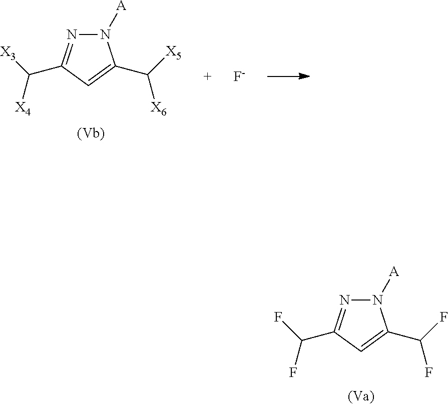

wherein A, X₃, X₄, X₅ and X₆ are as defined above.

In one embodiment, the process according to the invention comprises a least the following additional steps:
reacting compound of formula (III) with a fluoride anion source to obtain a compound of formula (IIIa),

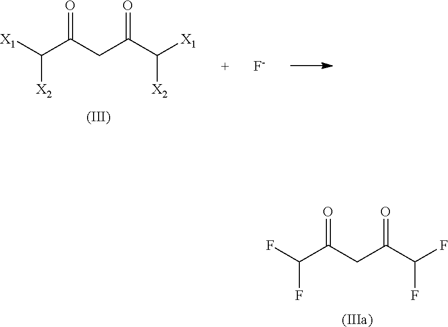

wherein X₁ and X₂, each independently are halogen, provided that at least one of X₁ and X₂ is not fluoro, and preferably X₁ and X₂, each independently are bromo or chloro.
reacting compound of formula (IIIa) with a compound of formula (IV) to obtain a compound of formula (Va):

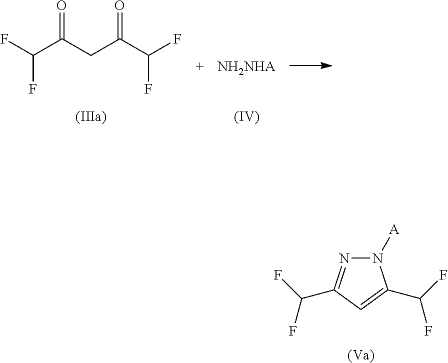

wherein A is as defined above for the compound of formula (V).

Alternatively, in one embodiment, the process according to the invention, comprises the following additional steps:
reacting a compound of formula (III) with a fluoride anion source to obtain one or several compounds of formula (III),

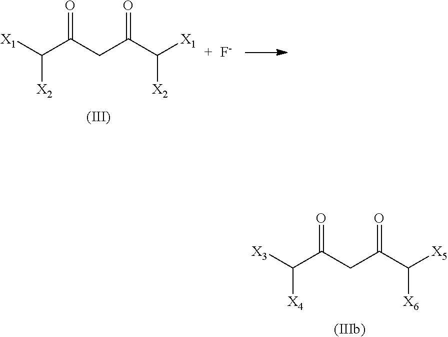

wherein X₁ and X₂, each independently are halogen, provided that at least one of X₁ and X₂ is not fluoro, and preferably X₁ and X₂, each independently are bromo or chloro,
X₃, X₄, X₅ and X₆ each independently are halogen, provided that at least one of X₃, X₄, X₅ and X₆ is fluoro,
reacting compounds of formula (IIIb) one or several times, and preferably 1, 2, 3 or 4 times with a fluoride anion source to obtain a compound of formula (IIIa),

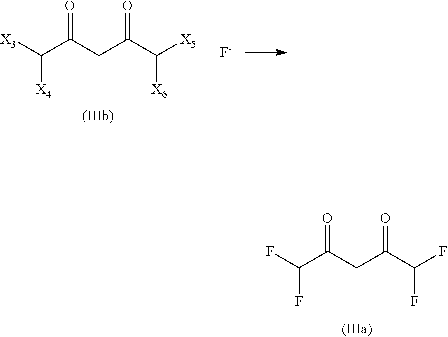

Wherein X₃, X₄, X₅ and X₆ are as defined above,
reacting compound of formula (IIIa) with a compound of formula (IV) to obtain a compound of formula (Va):

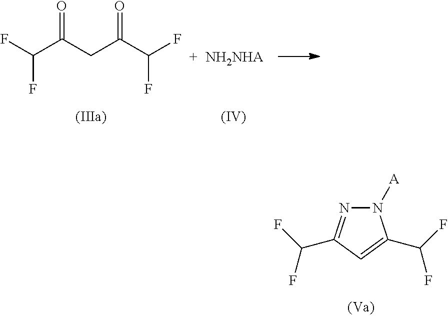

wherein A is as defined above for the compound of formula (V).

Alternatively, in one embodiment, the process according to the invention comprises the following additional step:
reacting a compound of formula (III) with a fluoride anion source to obtain one or several compounds of formula (IIIb),

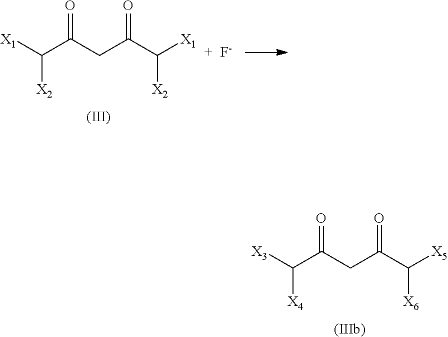

wherein X₁ and X₂, each independently are halogen, provided that at least one of X₁ and X₂ is not fluoro, and preferably X₁ and X₂, each independently are bromo or chloro,
X₃, X₄, X₅ and X₆ each independently are halogen, provided that at least one of X₃, X₄, X₅ and X₆ is fluoro,
reacting compound of formula (IIIb) with a compound of formula (IV) to obtain a compound of formula (Vb):

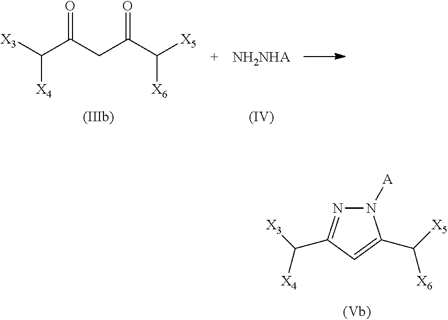

wherein A, X₃, X₄, X₅ and X₆ are as defined above,
reacting compounds of formula (Vb) one or several times, and preferably 1, 2, 3 or 4 times with a fluoride anion source to obtain a compound of formula (Va),

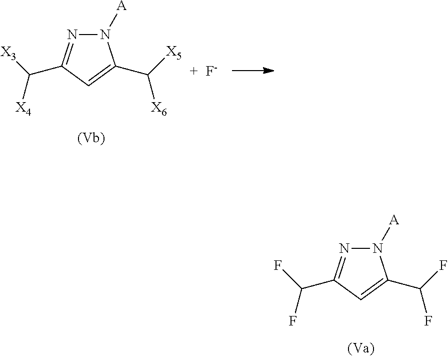

wherein A, X₃, X₄, X₅ and X₆ are as defined above.

A fluoride anion source is preferably a metal fluoride or HF and more preferably HF, NaF, KF, or CsF, most preferably KF or HF. Metal fluorides can be used alone or in combination with quaternary ammonium salts of formula (R₁)₄N⁺F⁻ where R₁ is C₁-C₆alkyl or quaternary phosphonium salts of formula (R₂)₄P⁺F⁻ where R₂ is substituted aryl and most preferably phenyl. HF can be used alone or as a complex with amines or pyridine.

In one embodiment of the process according to the invention, the substituent “A” on compounds of formula (V), (Va) and (Vb) can be replaced by a substituent “B” by reacting compounds of formula (V), (Va) or (Vb) with a compound of formula (VI)

B-X₇ (VI)
Wherein B is C₁-C₆alkyl, C₁-C₆haloalkyl, C₁-C₆alcoxycarbonyl C₁-C₆alkyl, hydroxycarbonyl C₁-C₆alkyl, C₁-C₆alkenyl, C₁-C₆alkynyl, —C(═O)R^{B}, C₁-C₆alkylaryl, and
R^{B} is hydrogen, C₁-C₆alkyl, C₁-C₆alcoxy, C₁-C₆haloalkyl, NH₂, C₁-C₆alkylamino, C₁-C₆dialkylamino or one of the following groups:

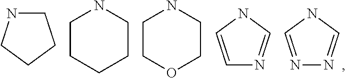

and
X₇ is halogen and preferably chloro, bromo or iodo.

For example, when A is hydrogen, compound (V) is reacted with a compound of formula (VI) to obtain a compound of formula (Vc):

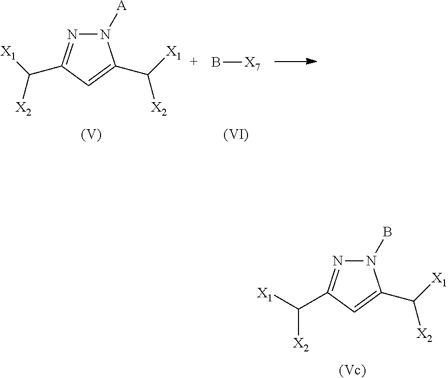

wherein A, B, X₁, X₂ and X₇ are as defined above.

Preferably, the first step of the process according to the invention is carried out under an inert gas atmosphere in a solvent selected from a group consisting of haloalkanes, substituted aromatic solvents such as nitroarenes, haloarenes, alkylarenes, alkoxyarenes, dialkyl ethers, tetrahydrofuran, dioxane. Most preferably haloalkanes or nitroarenes.

Compounds (Va) and (Vb) can similarly be reacted with a compound of formula (VI).

Preferably, the second step is carried out in a solvent selected from a group consisting of alcoholic solvents such as methanol or ethanol, haloalkanes, tetrahydrofuran or water using the reagent as a free base or as a salt in a combination with basic reagents such as pyridine, trialkylamines, alkali metal carbonates (Na₂CO₃, K₂CO₃) or hydroxides, for example LiOH, NaOH or KOH.

Preferably, the Lewis acid is selected from a group consisting of AlCl₃, SnCl₄, TiCl₄, SbCl₅, BF₃.Et₂O.

The molar ratio of compound of formula II to compound of formula I is between 2:1 and 4 to 1, preferably from 2:1 to 3:1. It is advantageous to conduct the reaction at dilution between 0.1 M to 1 M, preferably 0.3 M to 0.5 M of compound of formula II.

The reaction temperature is preferably between 0° C. to 150° C., more preferably between 40 and 100° C. and the reaction time is usually between 1 and 12 hours, preferably between 2 and 6 hours.

The process according to the invention can be carried out under standard pressure or under slightly elevated or reduced pressure. Typically, the reaction is run under standard pressure.

During the process according to the invention, and as shown above, one or several compounds of formula (III), (IIIa), (V) and (Vb) can be obtained. These intermediates compounds are part of this invention

The present invention also includes compounds of formula (III):

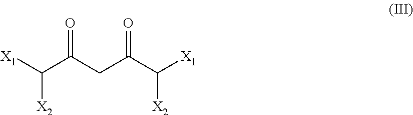

wherein X₁ and X₂ each independently are halogen, preferably bromo, chloro or fluoro, and most preferably X₁ and X₂ are chloro.

The invention also relates to compound of formula (IIIb):

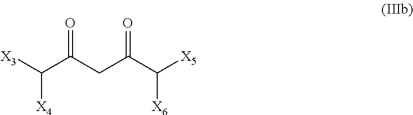

wherein X₃, X₄, X₅ and X₆ each independently are halogen, and at least one of X₃, X₄, X₅ and X₆ is fluoro.

Non limiting examples of these intermediates are also part of this invention and disclosed below as compounds of formula (X), (Y) and (Z):

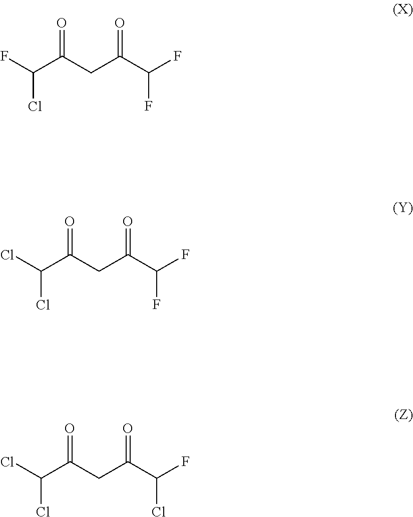 [SMILES: O=C(CC(=O)C(F)Cl)C(F)F ; O=C(CC(=O)C(Cl)Cl)C(F)F ; O=C(CC(=O)C(Cl)Cl)C(F)Cl]

The invention also includes compounds of formula (V):

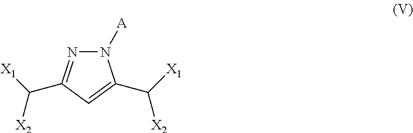

wherein A is hydrogen, C₁-C₆alkyl, C₁-C₆haloalkyl, C₁-C₆alcoxycarbonyl C₁-C₆alkyl, hydroxycarbonyl C₁-C₆alkyl, C₁-C₆alkenyl, C₁-C₆alkynyl, —C(═O)R^{A}, C₁-C₆alkylaryl.
R^{A} is hydrogen, C₁-C₆alkyl, C₁-C₆alcoxy, C₁-C₆haloalkyl, NH₂, C₁-C₆alkylamino, C₁-C₆dialkylamino or one of the following groups:

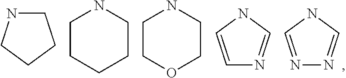

and X₁ and X₂ are chloro.

Preferably, for compounds of formula (V):
X₁ and X₂ are chloro and -A is —CH₂CO₂R, with R being hydrogen or C₁-C₄ alkyl, and preferably methyl or ethyl.

The present invention also includes compounds of formula (Vb):

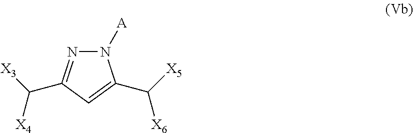

wherein A is hydrogen, C₁-C₆alkyl, C₁-C₆haloalkyl, C₁-C₆alcoxycarbonyl C₁-C₆alkyl, hydroxycarbonyl C₁-C₆alkyl, C₁-C₆alkenyl, C₁-C₆alkynyl, —C(═O)R^{A}, C₁-C₆alkylaryl.
R^{A} is hydrogen, C₁-C₆alkyl, C₁-C₆alcoxy, C₁-C₆haloalkyl, NH₂, C₁-C₆alkylamino, C₁-C₆dialkylamino or one of the following groups:

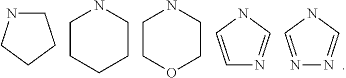

X₃, X₄, X₅ and X₆ each independently are halogen, provided that at least one and no more than three of X₃, X₄, X₅ and X₆ is fluoro.

Preferably, in compounds of formula (Vb),

A is —CH₂CO₂R, with R being hydrogen or C₁-C₄ alkyl,

X₃, X₄, X₅ and X₆ each independently are halogen, provided that at least one and no more than three of X₃, X₄, X₅ and X₆ is fluoro.

Non limiting examples of these intermediates are also part of this invention and disclosed below as compounds of formula (X), (Y) and (Z):

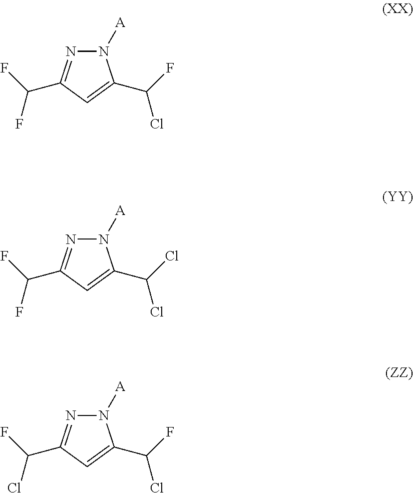

### EXAMPLE 1

### Preparation of 3,5-bis(dichloromethyl)-1H-pyrazole

### Step 1. 1,1,5,5-tetrachloropentane-2,4-dione

Aluminium chloride (5.8 g, 44 mmol) was placed in a two-necked RB flask equipped with a condenser and a drying tube. The flask was purged with N₂ followed by the addition of nitrobenzene (5.0 mL) and dichloroethane (10.0 mL). The mixture was stirred until all aluminium chloride was dissolved, leaving a brown solution. Pentane-2,4-dione (4.4 g, 44 mmol) was then added dropwise (5 min). The reaction mixture was cooled to 0° C. by placing the flask in an ice-bath and 2,2-dichloroacetyl chloride (19.0 g, 130 mmol) was added dropwise over 5 min. The reaction mixture was then removed from ice bath and heated to 65° C. for 5 h. The reaction mixture was cooled and slowly poured into a flask containing conc. HCl (15 mL) and ice (120 g), followed by stirring overnight. The contents of the flask were extracted with DCM (3×50 mL), washed with water, the organic layers were dried over anhydrous Na₂SO₄ and evaporated under reduced pressure. The residue was purified by vacuum distillation (105° C. and 2×10⁻³ mbar) to give 1,1,5,5-tetrachloropentane-2,4-dione (9.5 g, 91%) as a pale yellow oil.

1H NMR (400 MHz, CDCl₃) δ 6.42 (s, 1H), 6.0 (s, 2H)

### Step 2. 3,5-bis(dichloromethyl)-1H-pyrazole

To a solution of 1,1,5,5-tetrachloropentane-2,4-dione (1.0 g, 4.2 mmol) in ethanol (10 mL) was slowly added hydrazine monohydrate (1.03 eq., 4.3 mmol) at rt over a period of 5 min. The reaction mixture was stirred at rt for 16 h, poured onto ice (25 g) and extracted with DCM (3×50 mL). The combined organic layers were dried on Na₂SO₄ and concentrated to give 3,5-bis(dichloromethyl)-1H-pyrazole (0.80 g, 81% yield) as yellow semisolid mass.

1H NMR (400 MHz, CDCl₃) δ 7.50 (s, 2H), 6.64 (s, 1H)

### EXAMPLE 2

### Preparation of Ethyl 2-[3,5-bis(dichloromethyl)pyrazol-1-yl]acetate

To a solution of 1,1,5,5-tetrachloropentane-2,4-dione (500 mg, 2.1017 mmol) in ethanol (5 mL), ethyl hydrazinoacetate hydrochloride (336 mg, 2.1648 mmol) was added at 0° C. The reaction mixture was stirred at 0° C. for 15 min, ice-bath removed and stirring continued at rt for 16 h. It was poured into ice water (30 mL), aqueous layer (pH=1-2) was neutralized using solid NaHCO₃ and extracted with DCM (3×50 mL). The combined organic layers were dried on Na₂SO₄ and concentrated to give crude compound (630 mg) which was purified by silica gel chromatography (8% ethyl acetate in cyclohexane) to afford ethyl 2-[3,5-bis(dichloromethyl)pyrazol-1-yl]acetate as colorless oil (340 mg, 50%)

1H NMR (400 MHz, CDCl₃) δ 6.81 (s, 1H), 6.78 (s, 1H), 6.72 (s, 1H), 5.09 (s, 2H), 4.29 (dd, J=7.2 Hz, 2H), 1.31 (t, J=7.2 Hz, 3H).

### EXAMPLE 3

### Preparation of 3,5-bis(difluromethyl)-1H-pyrazole

3,5-bis(dichloromethyl)-1H-pyrazole (233 mg, 1.00 mmol) was placed in a polytetrafluoroethylene flask and dissolved in triethylamine trihydrofluoride (2.0 ml, 12 mmol). The resulting solution was purged with dry argon and stirred at 150 C for 2 h.

The reaction mixture was cooled to ambient temperature and poured into ice water. The mixture was extracted with ethyl acetate (2×10 ml), the organic phase was washed with aq saturated NaHCO₃ and combined organic layers were dried over anhydrous MgSO₄. Concentration of the solution provided 3,5-bis(difluoromethyl)-1H-pyrazole as a pale yellow oil (109 mg, 65%).

1H NMR (400 MHz, CDCl₃) δ 7.58 (s, 1H), 6.79 (t, J=56 Hz, 2H), 6.78 (s, 1H)

### EXAMPLE 4

### Preparation of ethyl 2-[3,5-bis(difluoromethyl)pyrazol-1-yl]acetate

2-[3,5-bis(dichloromethyl)pyrazol-1-yl]acetate (500 mg, 1.56 mmol) was placed in a polytetrafluoroethylene coated reactor and dissolved in triethylamine trihydrofluoride (2.0 ml, 12 mmol). The resulting mixture was stirred at 150 C for 16 h. The reaction mass was cooled to ambient temperature and poured into ice cold water (100 ml). The mixture was neutralized by adding NaHCO₃ and extracted with DCM (3×80 ml). Combined organic layers were dried over anhydrous Na₂SO₄ and evaporated under reduced pressure. The crude product was purified by silica gel chromatography (5% EtOAc in cyclohexane) to provide 2-[3,5-bis(difluoromethyl)pyrazol-1-yl]acetate (115 mg, 29%) as a fluffy solid.

1H NMR (400 MHz, CDCl₃) δ 6.79 (t, J=54.3 Hz, 1H), 6.76 (s, 1H), 6.67 (t, J=54.7 Hz, 1H), 5.05 (s, 2H), 4.26 (q, J=7.3 Hz, 2H), 1.29 (t, J=7.3 Hz, 3H)

1. A process comprising:
reacting a compound of formula (I), with a compound of formula (II), in the presence of a Lewis acid to obtain a compound of formula (III): 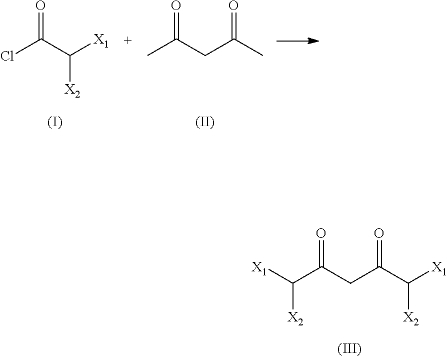
wherein X₁ and X₂ each independently are halogen, then
reacting the compound of formula (III) with a compound of formula (IV) to obtain a compound of formula (V): 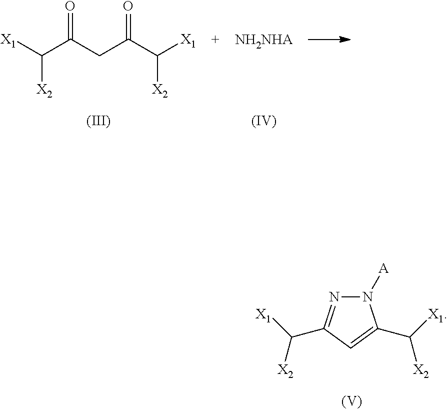
wherein,
A is hydrogen, C₁-C₆alkyl, C₁-C₆haloalkyl, C₁-C₆alcoxycarbonylC₁-C₆alkyl, hydroxycarbonylC₁-C₆alkyl, C₁-C₆alkenyl, C₁-C₆alkynyl, —C(═O)R^{A}, C₁-C₆alkylaryl, and
R^{A} is hydrogen, C₁-C₆alkyl, C₁-C₆alcoxy, C₁-C₆haloalkyl, NH₂, C₁-C₆alkylamino, C₁-C₆dialkylamino or one of the following groups: 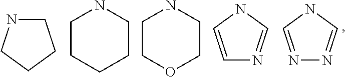
and X₁ and X₂ each independently are halogen.

2. A process according to claim 1, wherein X₁ and X₂ each independently are bromo, chloro or fluoro.

3. A process according to claim 1, wherein X₁ and X₂ each independently are chloro or fluoro.

4. A process according to claim 1, wherein A is —CH₂CO₂R, with R being hydrogen or C₁-C₄ alkyl.

5. A process according to claim 1, comprising at least the following additional step:
reacting a compound of formula (V) with a fluoride anion source, to obtain a compound of formula (Va): 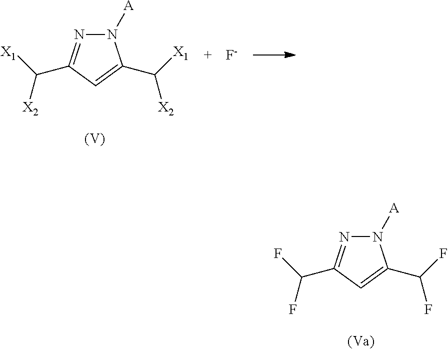
wherein A is as defined in claim 1,
X₁ and X₂, each independently are halogen, provided that at least one of X₁ and X₂ is not fluoro.

6. A process according to claim 1 comprising at least the following additional steps:
reacting compound of formula (III) with a fluoride anion source to obtain a compound of formula (IIIa), 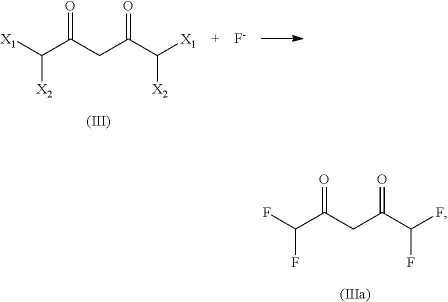
Wherein X₁ and X₂, each independently are halogen, provided that at least one of X₁ and X₂ is not fluoro,
reacting compound of formula (IIIa) with a compound of formula (IV) to obtain a compound of formula (Va): 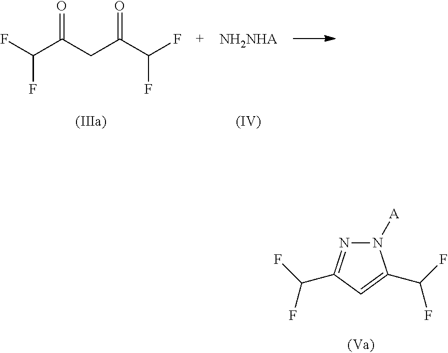
wherein A is as defined in claim 1.

7. A process according to claim 1 wherein, when A is hydrogen, compound (V) is reacted with a compound of formula (VI) to obtain a compound of formula (Vc): 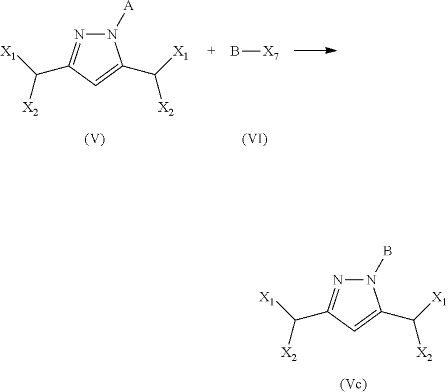
Wherein, A is as defined in claim 1,
B is C₁-C₆alkyl, C₁-C₆haloalkyl, C₁-C₆alcoxycarbonyl C₁-C₆alkyl, hydroxycarbonyl C₁-C₆alkyl, C₁-C₆alkenyl, C₁-C₆alkynyl, —C(═O)R^{B}, C₁-C₆alkylaryl, and
R^{B} is hydrogen, C₁-C₆alkyl, C₁-C₆alcoxy, C₁-C₆haloalkyl, NH₂, C₁-C₆alkylamino, C₁-C₆dialkylamino or one of the following groups: 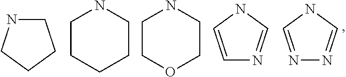
X₁ and X₂ are as defined in claim 1,
X₇ is halogen and preferably chloro, bromo or iodo.

8. A process according to claim 1, wherein the first step is carried out under an inert gas atmosphere in a solvent selected from a group consisting of a haloalkane, a substituted aromatic solvent, and a nitroarene.

9. A process according to claim 1, wherein the second step is carried out in a solvent selected from a group consisting of an alcoholic solvent, a haloalkane, tetrahydrofuran and water.

10. A compound of formula (III): 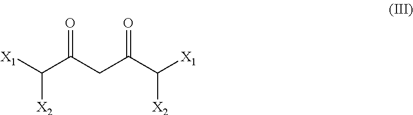
wherein X₁ and X₂ each independently are halogen.

11. A compound according to claim 10, wherein X₁ and X₂ each independently are bromo, chloro or fluoro.

12. A compound according to claim 10, wherein X₁ and X₂ are chloro.

13. A compound of formula (IIIb): 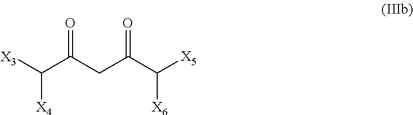
wherein X₃, X₄, X₅ and X₆ each independently are halogen, and at least one of X₃, X₄, X₅ and X₆ is fluoro.

14. A compound of formula (V): 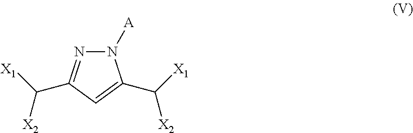
wherein A is hydrogen, C₁-C₆alkyl, C₁-C₆haloalkyl, C₁-C₆alcoxycarbonyl C₁-C₆alkyl, hydroxycarbonyl C₁-C₆alkyl, C₁-C₆alkenyl, C₁-C₆alkynyl, —C(═O)R^{A}, C₁-C₆alkylaryl.
R^{A} is hydrogen, C₁-C₆alkyl, C₁-C₆alcoxy, C₁-C₆haloalkyl, NH₂, C₁-C₆alkylamino, C₁-C₆dialkylamino or one of the following groups: 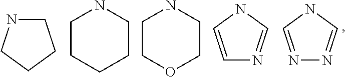
and X₁ and X₂ are chloro; or
a compound of formula (Vb): 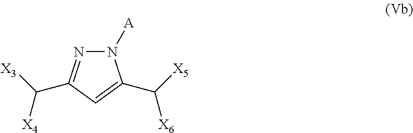
wherein A is hydrogen, C₁-C₆alkyl, C₁-C₆haloalkyl, C₁-C₆alcoxycarbonyl C₁-C₆alkyl, hydroxycarbonyl C₁-C₆alkyl, C₁-C₆alkenyl, C₁-C₆alkynyl, —C(═O)R^{A}, C₁-C₆alkylaryl,
R^{A} is hydrogen, C₁-C₆alkyl, C₁-C₆alcoxy, C₁-C₆haloalkyl, NH₂, C₁-C₆alkylamino, C₁-C₆dialkylamino or one of the following groups: 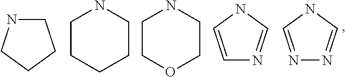
X₃, X₄, X₅ and X₆ each independently are halogen, provided that at least one and no more than three of X₃, X₄, X₅ and X₆ is fluoro.

15. (canceled)
